(19) United States
(12) Patent Application Publication
(10) Pub. No.: US 2026/0083385 A1
(43) Pub. Date: Mar. 26, 2026

(54) SYSTEMS AND METHODS FOR ANALYZING ELECTROENCEPHALOGRAMS

(71) Applicant: BrainMaster Technologies, Inc., Bedford, OH (US)

(72) Inventors: Thomas Collura, Chagrin Falls, OH (US); Agostino Roseace, Chardon, OH (US)

(21) Appl. No.: 19/338,510

(22) Filed: Sep. 24, 2025

Related U.S. Application Data
(60) Provisional application No. 63/698,176, filed on Sep. 24, 2024.

(51) Int. Cl. A61B 5/372; A61B 5/00; A61B 5/375; A61B 5/384

(52) U.S. Cl. CPC A61B 5/372; A61B 5/375; A61B 5/384; A61B 5/7257

(57) ABSTRACT

Disclosed are systems and methods for analyzing and evaluating electroencephalogram (EEG) signals, comprising built-in independent component analysis (ICA), frequency-domain averaging, standardized Low Resolution Electromagnetic Tomography (sLORETA), and source localization to deconvolve EEG rhythms into individual sources and patterns. The disclosed systems and methods capture the morphology of one “event” being produced by that brain source and analyze the pattern of “events” over time. The detection and isolation of singular events and the association of the events to the particular times that they occur can allow insight into the morphology of the waves, and the exact timing of the brain events, including effects that change across time.

### CROSS-REFERENCE TO RELATED APPLICATIONS

The present application claims the benefit of U.S. Provisional Patent Application No. 63/698,176 filed on September 24, 2024, the entirety of which is incorporated by reference herein.

### BACKGROUND

Aspects described herein relate to systems and methods for analyzing and evaluating electroencephalogram (EEG) signals and, more particularly, to systems and methods that can deconvolve EEG signals into discrete time points and individual morphological brain events.

Electroencephalograms (EEGs) can record and measure electrical impulses generated by a brain using a plurality of non-invasive sensors. The plurality of sensors may be positioned across the scalp, for example, to detect transcranial brainwave patterns. Captured electrical signals are amplified, processed, and converted into meaningful data, providing insights into brain activity for clinical diagnoses, therapy, and neurological research.

### BRIEF SUMMARY

According to a first embodiment described herein, a method of analyzing electroencephalogram signals, comprising capturing the electroencephalogram (EEG) signals from a plurality of sensors, performing independent component analysis (ICA) on the electroencephalogram signals to provide ICA-refined electroencephalogram data, performing standardized Low Resolution Electromagnetic Tomography (sLORETA) on the ICA-refined electroencephalogram data, computing successive Fast Fourier Transforms (FFT) of epochs and averaging FFT amplitudes of the epochs to provide an averaged power spectrum, computing an inverse FFT on the averaged power spectrum and using autocorrelation to find matches with the electroencephalogram signals, determining a plurality of events based on matches of the autocorrelation and the electroencephalogram signals.

In various embodiments of the above example, the method further comprises displaying the plurality of determined events, performing ICA produces a plurality of individual components, each of the plurality of individual components has a complete frequency spectrum and an amplitude for every channel of the electroencephalogram signals, displaying the plurality of components, where the display includes a percentage of total EEG energy contained in the component, a surface site at which it is maximum, a sLORETA localized lobe, a brain region, a Brodmann area, and a FFT peak energy for each component of the plurality of components, providing neuro-feedback based on the determined plurality of events, and the neuro-feedback is based on the shape of the determined plurality of events.

According to another example of the present disclosure, a system for analyzing electroencephalogram signals, comprises one or more computer memory units for storing computer instructions; and one or more processors operatively coupled to the one or more computer memory units, the one or more processors configured to perform the operations of: capturing the electroencephalogram signals from a plurality of sensors, performing independent component analysis (ICA) on the electroencephalogram signals to provide ICA-refined electroencephalogram data, performing standardized Low Resolution Electromagnetic Tomography (sLORETA) on the ICA-refined electroencephalogram data, computing successive Fast Fourier Transforms (FFT) of epochs and averaging FFT amplitudes of the epochs to provide an averaged power spectrum, computing an inverse FFT on the averaged power spectrum and using autocorrelation to find matches with the electroencephalogram signals, and determining a plurality of events based on matches of the autocorrelation and the electroencephalogram signals.

In various embodiments of the above example, the system further comprises a display configured to display the plurality of determined events, performing ICA produces a plurality of individual component, each of the plurality of individual components has a complete frequency spectrum and an amplitude for every channel of the electroencephalogram signals, the display is configured to display the plurality of individual components in a graph or table, the graph or table includes a percentage of total EEG energy contained in the component, a surface site at which it is maximum, a sLORETA localized lobe, a brain region, a Brodmann area, and a FFT peak energy for each component of the plurality of components, an output configured to provide neuro-feedback based on the determined plurality of events, and the neuro-feedback is based on the shape of the determined plurality of events.

The following description and the related drawings set forth in detail certain illustrative features of one or more aspects of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present teachings may be better understood by reference to the following detailed description taken in connection with the following illustrations, in which like reference characters refer to like parts throughout

FIG. 1 is a flow chart of an example embodiment of a method for analyzing EEG recordings in accordance with aspects disclosed herein.

FIG. 2 is a block diagram of an example embodiment of an operating environment within which aspects of the subject disclosure can be performed to analyze EEG recordings.

FIGS. 3-4 are graphical representations showing analyses related to the example application of an independent component analysis (ICA) on an EEG recording.

FIG. 5 is a list of components and characteristics of the components used in the example application of an independent component analysis (ICA) on an EEG recording.

FIG. 6-9 are graphical representations showing analyses related to the example application of the ICA decomposition for selected components.

FIG. 10 is a graphical representation showing additional data that can be realized through the ICA by classifying each component by type.

FIG. 11 is a graphical representations showing characteristics from a database of EEGs that were analyzed by a board certified clinical neurophysiologist and QEEG Diplomates.

FIGS. 12-13 are graphical representations showing a qualitative estimation of how the disclosed systems and methods analyze EEGs and they compare to the expectations of a visual quality review by a clinical neurophysiologist.

FIG. 14 is a flow chart of an example embodiment of a method for analyzing EEG recordings in accordance with aspects disclosed herein.

FIGS. 15-16 are graphical representations showing power spectrums associated with wavelets and analysis thereof in accordance with aspects disclosed herein.

FIGS. 17-20 are graphical representations showing pulses and pulse trains and analysis thereof in accordance with aspects disclosed herein.

FIG. 21 is a graphical representation of the example model event and analysis thereof in accordance with aspects disclosed herein.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Aspects described herein provide apparatuses, methods, processing systems, and computer-readable mediums associated with Electroencephalograms (EEGs), which record and measure electrical impulses generated by a brain.

Electroencephalograms are conventionally analyzed as a whole and understood as consisting of frequencies generated by free-running oscillators, not as a multitude of discrete and singular events distributed in time. In an EEG recording, for example, the overall pattern of brain activity is typically examined by observing the collective behavior of the electrical waves produced by the brain over a period of time. The EEG recording generally provides a comprehensive view of brain activity displayed as a series of waveforms, which represent electrical activity detected by the electrodes placed on the scalp. Efforts to decompose EEGs to smaller evaluative components have been met with limited success for various reasons. Including, for example, the fact that it is difficult to decompose EEGs into smaller components due to the effects of volume conduction and the presence of many generators at a single time. Additionally, conventional systems and methods for analyzing EEGs are not able to detect smaller, discrete signals within the EEGs, and thus, are not able to analyze EEGs apart from the whole. Further, some do not believe breaking down EEGs into smaller component parts is worth the effort or would provide useful information.

Moreover, quantitative EEGs (QEEGs), which involve the recording and analysis of digital EEG signals sometimes referred to as brain mapping, are like a “blender” that analyzes the entire record without regard to morphology or individualized components of the EEG. QEEGs break EEGs into “frequency bands” that have predefined ranges and analyzes the entire EEG recording as one large sample, albeit broken into segments called “epochs.”

QEEGs, like their EEG counterparts, suffer the same analysis shortcomings. Both, for example, may have limited spatial resolution preventing the precise pinpointing of exact locations of brain activity. Particularly, the electrical signals detected by scalp electrodes can be influenced by the volume conduction properties of the skull and scalp tissues, which can blur the spatial localization of neural activity. Both QEEGs and EEGs may also have limited depth sensitivity, primarily detecting electrical activity from the outer layers of the brain (cortex) and are less sensitive to activity occurring deeper within the brain structures. Recordings may also be influenced by various artifacts, such as muscle movements, eye blinks, and environmental interference, which may obscure the underlying brain activity. Distinguishing between genuine brain signals and artifacts requires careful signal processing and interpretation. Additionally, while recording may provide high temporal resolution capturing changes in brain activity with millisecond precision, QEEGs and EEGs do not provide detailed information about the temporal sequence of neural events occurring within the brain. Finally, the QEEG and EEG signals reflect the aggregate activity of large populations of neurons and do not provide detailed information about the specific neural circuits or neurotransmitter systems involved in brain function. The lack of detailed information about the neural circuits or neurotransmitter systems makes it challenging to infer the underlying mechanisms of observed EEG patterns accurately.

The disclosed systems and methods overcome these shortcomings and are able to analyze EEGs to detect, isolate, attribute, and evaluate the discrete events within the EEGs distributed in time. The disclosed systems and methods are able to deconvolve resting EEG sources into events and time points, revealing the underlying discrete time structure. The disclosed systems and methods comprise first applying independent components analysis (ICA) to remove the effects of volume conduction, and then using a frequency-domain deconvolution. The disclosed systems and methods isolate the morphology of individual brain events and can reconstruct the exact time points at which they occur. The detailed time statistics of each component can reveal the pattern of subcortical spiking that elicits each brain event. While conventional analysis views the entire record without being able to discern morphology, the disclosed systems and methods have the precision to identify constituent and singular events, dismantle constituent and singular events, and associate the constituent and singular events with the time in which they occur. Neuro-feedback can be provided to a patient based on these events and morphology.

The disclosed systems and methods are able to determine the morphology of the waves and the exact timing of the waves, including effects that change across time. The disclosed systems and methods first decompose the signal into its apparent volume-conducted sources and then further process these components using frequency-domain averaging to produce an estimate of each event wave. The disclosed systems and methods further match the signatures against the measured component to determine the most likely times or “instants” for the occurrence of each event. These time-point series can provide statistical information regarding the point process that defines the occurrence of these brain events. For example, if it is determined that the point-process is highly regular, then a prominent frequency band may emerge, as well as harmonics reflecting the energy at all frequencies. Otherwise, if point-process is determined to be less regular, the energy will be more smoothly spread across the range. Some often visible components are eye blinks, eye movement, EKG artifact, blood-volume pulse, and similar physiological yet not-brain sources. Remaining sources reflect the commonly recognized sources (posterior alpha, midline theta, etc.), and show additional detail, e.g., multiple PDR sources or complex temporal sources.

The disclosed systems and methods can separate and understand multiple sources in the EEGs, including posterior dominant rhythms, general alpha events, midline theta events, and various temporal and other generators. Each component exists at all channels, and contains all possible frequencies, but are not to be confused with channels or with frequencies. This type of deconvolution provided by the disclosed systems and methods reveals the underlying generators in a manner that is consistent with the deep mechanisms, avoiding the misconceptions that arise with a simple frequency/oscillator model of conventional EEG analysis. The disclosed systems and methods validates the determinations by identifying known sources, via the first standardized Low Resolution Electromagnetic Tomography (sLORETA) projection. In addition, after deconvolution, more precise timing becomes evident, along with a more detailed view of each transient component. These are found to be not just simple bursts of a single frequency, but are complex transients that encompass the entire EEG frequency range, while also exhibiting peaks, and ripples, that reflect the morphology of the single events, which are recovered as “signatures.”

The disclosed systems and methods do not depend on a database for its operation, because the systems and methods are based on first principles. The ICA decomposes the EEG into a set of sources (dipoles) that are synchronous in time, and are seen to varying degrees within any channel. This implicitly seeks signals which are volume conducted, by estimating the amplitude at each scalp location. For this reason, the mixing matrix entries are by definition suitable for sLORETA projection. The mixing matrix contains the amplitudes and polarities of each component in each channel, thus reflecting the exact volume conduction distribution at every instant. By inverting each component matrix entries, an accurate estimate of the source of each component is created. Despite the fact that the decomposition and deconvolution are database free, a database can be constructed from the output data, showing which types of events and time patterns exist in the subject population and aiding in interpretation. For example, PDR sources are found variously distributed throughout the visual and association cortex, depending on the individual. Individual PDR sources are found to be stable within subject, thus reflecting potentially valuable individual characteristics.

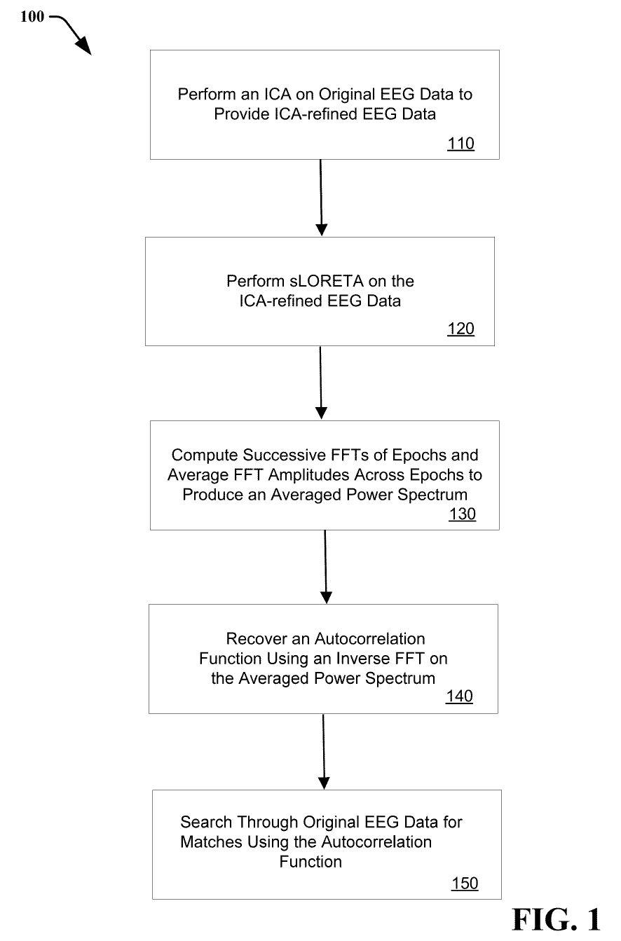

Turning to FIG. 1, disclosed is a method for analyzing and evaluating electroencephalograms (EEGs) 100, for example, as a European Data Format (EDF) file, EDF+ file, or live EEG data from a plurality of sensors. In one embodiment, the method 100 may comprise performing independent component analysis (ICA) on original EEG data to provide ICA-refined EEG data (step 110). For example, in one embodiment, the ICA step 110 may be used to remove eye activity, muscle movements, environmental interference, other signal artifacts or the like. After the ICA is performed on the original EEG data, the method 100 may further perform standardized Low Resolution Electromagnetic Tomography (sLORETA) on the ICA-refined EEG data (step 120). For example, in one embodiment, the sLORETA projection of the mixing matrix may be applied to each component of the ICA-refined data to estimate the source location and orientation. After performing sLORETA projections, the method computes successive Fast Fourier transforms (FFTs) of epochs of the ICA-refined EEG data to and averaging FFT amplitudes across epochs to produce an averaged power spectrum (step 130). In one embodiment, the epochs may be 10 second epochs across the entire EDF file. In some embodiments, the epochs can be shorter or longer, based on component characteristics. After the computation and averaging of step 130, the method includes recovering the autocorrelation function by using an inverse FFT on the averaged power spectrum (step 140). The method 100 further comprises searching through original EEG data for matches using the autocorrelation function (step 150). For example, in one embodiment, the matches between the autocorrelation function and the original EEG data may be marked as events and used for real-time feedback to user. In other embodiments, the file comprising the EEG data is annotated indicating such events and can be reviewed at a later date. The annotations can include user comments, component characteristics, and the like.

In one embodiment, the method of analyzing and evaluating EEGs 100 may be used to deconvolve EEG data into individual sources and patterns. For instance, the method 100 can be used to detect and isolate singular events. Using this detection and isolation of events and the association of the events to the particular times that they occur provide insight into the morphology of the waves and the exact timing of the brain events, including effects that change across time.

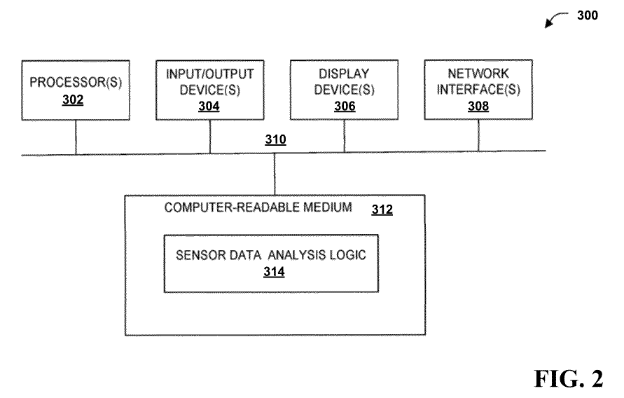

FIG. 2 depicts an example processing system 300 configured to perform various aspects described herein, including, for example, the method described above and external processing of sensor data from an EEG.

Processing system 300 is generally an example of an electronic device configured to execute computer-executable instructions, such as those derived from compiled or interpreted computer code, including without limitation personal computers, tablet computers, servers, smart phones, smart devices, wearable devices, augmented or virtual reality devices, and others.

In the depicted example, processing system 300 includes one or more processors 302, one or more input/output devices 304, one or more display devices 306, and one or more network interfaces 308 through which processing system 300 is connected to one or more networks ( e.g., a local network, an intranet, the Internet, or any other group of processing systems communicatively connected to each other), and computer-readable medium 312.

In the depicted example, the aforementioned components are coupled by a communication bus 310, which may generally be configured for data or power exchange amongst the components. The communication bus 310 may be representative of multiple buses, while only one is depicted for simplicity.

Processor(s) 302 are configured to retrieve and execute instructions stored in one or more memories, including local memories like the computer-readable medium 312, as well as remote memories and data stores, e.g., cloud based databases. Similarly, processor(s) 302 are configured to retrieve and store application data residing in local memories like the computer-readable medium 312, as well as remote memories and data stores. More generally, communication bus 310 is configured to transmit programming instructions and application data among the processor(s) 302, display device(s) 306, network interface(s) 308, and computer-readable medium 312. In certain embodiments, processor(s) 302 are included to be representative of one or more central processing units (CPUs), graphics processing units (GPUs), tensor processing units (TPUs), accelerators, and other processing devices.

Input/output device(s) 304 may include any device, mechanism, system, interactive display, or various other hardware components for communicating information between processing system 300 and a user of processing system 300. For example, input/output device(s) 304 may include input hardware, such as a keyboard, touch screen, button, microphone, or other device for receiving input from the user. Input/output device(s) 304 may further include display hardware, such as, for example, a monitor, a video card, or other device for sending or presenting visual data to the user. In certain embodiments, input/output device(s) 304 is or includes a graphical user interface.

In some embodiments, the input/output devices also include a plurality of sensors, e.g., EEG electrodes and sensors, or the like, and the processing system 300 can process data real-time. That is, in some embodiments the processing system 300 comprising a computer with an EEG sensor attached to a patient. During an EEG test or monitoring session, the processing system 300 can process and analyze the EEG data in real-time. The technician can annotate or comment on various parts of the data file displayed on a display device 306. For instance, the technician can input notes and comments at various parts of the EEG or in the file in general.

The input/output devices 304 can further include systems configured for notification or feedback. For example, the input/output devices 304 can include neuro-feedback devices that provide visual and/or auditory cues for a user, using these cues, the individual can learn to control brain waves and produce desired changes in various cognitive, emotional, and behavioral issues. In some embodiments, the input/output devices 304 can include a buzzer or an alarm configured to signal in a predetermined case, e.g., for neuro-feedback, if the EEG is not connected properly, or if there is an error with a data file. Additionally, the processing system 300 is configured to notify the user or patient of events in deconvoluted signals. For example, the processing system 300 can utilize a machine learning model or artificial intelligence model that is trained to detect specific neurological conditions based on the deconvoluted signals and configured to provide feedback if a signal suggests a specific condition, e.g., seizures, epilepsy, brain tumors, head injuries, inflammation, sleep disorders, dementia, confusion, or the like. For instance, a pop-up or a flashing notification can be displayed on the display 306. In other instances, the processing system 300 can use a trained machine learning model to analyze the singular components, morphology, and/or provide neuro-feedback.

Display device(s) 306 may generally include any device configured to display data, information, graphics, user interface elements, and the like to a user. For example, display device(s) 306 may include internal and external displays, such as an internal display of a tablet computer or an external display for a server computer or a projector. Display device(s) 306 may further include displays for devices, such as augmented, virtual, or extended reality devices.

Network interface(s) 308 provide processing system 300 access to external networks and processing systems. Network interface(s) 308 can generally be any device capable of transmitting or receiving data through a wired or wireless network connection. Accordingly, network interface(s) 308 can include a transceiver for sending or receiving wired or wireless communication. For example, Network interface(s) 308 may include an antenna, a modem, a LAN port, a Wi-Fi card, a WiMAX card, cellular communications hardware, near-field communication (NFC) hardware, satellite communication hardware, or any wired or wireless hardware for communicating with other networks or devices/systems. In certain embodiments, network interface(s) 308 includes hardware configured to operate in accordance with the Bluetooth® wireless communication protocol.

Computer-readable medium 312 may be a volatile memory, such as a random access memory (RAM), or a non-volatile memory, such as non-volatile random access memory, phase change random access memory, or the like. In this example, computer-readable medium 312 includes sensor data analysis logic 314. The sensor data analysis logic 314 can be performed by the flexible circuit board or external processing device.

Note that FIG. 2 is just one example of a processing system consistent with aspects described herein, and other processing systems having additional, alternative, or fewer components are possible consistent with this disclosure.

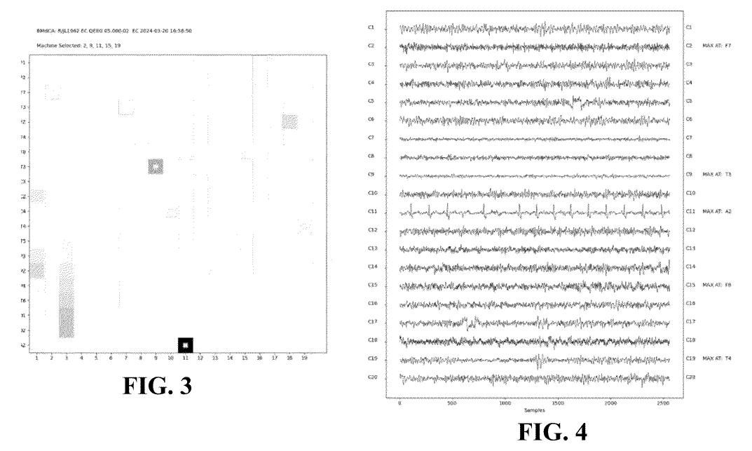

In one embodiment, the system 300 of FIG. 2 executes a program stored on the computer-readable medium 312, where the program comprises the method of FIG. 1. The system 300 provides an automated screening of EEGs by providing a guided experience for the user. In one embodiment, the system 300 walks through the steps of the FIG. 1 with the user in an automated fashion, allowing a user to provide visual inspection. In other embodiments, the system 300 is given EEG signals and the system performs the methods disclosed herein to generate a report. For example, a report can comprise of graphs, images, tables, and the like, similar to the illustrations of FIGS. 3-13. This report can be printed, displayed, emailed, or otherwise disturbed by the system using the input/output devices 304, display devices 306, and network interfaces 308.

In some embodiments, the reports provide analysis of “moments” of the EEG which reflect individual components and characteristics of those components. For instance, the reports can illustrate the component as a waveform and/or numerical values for each characteristic.

In some embodiments, a machine learning (ML) model or artificial intelligence (AI) model is trained to classifying and/or recognize the components based on the method disclosed herein. The processing system 300 can further include such an ML model trained to recognize components, where real-time data is input into the ML model, and the ML model outputs classified components. For example, an ML model can be trained using the databases, methods, or the like described herein to provide real-time analysis of EEG signals. The ML model can be trained using supervised training, non-supervised training, or semi-supervised training. The ML model can be trained on patient data or generated data.

FIGS. 3-4 show analyses related to the application of an independent component analysis (ICA) on an EEG recording. In an embodiment, the ICA produces a set of individual components C1-C20 that are mathematically isolated using an iterative procedure. For example, in one embodiment, the ICA converts the 19 channels of the EEG recording into 19 components (20 components if the ear reference signal is included) which are signals that are distributed across the channels in a simple relationship. In some embodiments, the ICA removes the effects of volume conduction and converts the mixed signals from the scalp in the EEG recording into a set of estimated sources. Each source has a complete frequency spectrum shown in FIG. 4. These sources are not channels or frequencies. Instead, each source has its own amplitude for every channel (shown in the mixing matrix of FIG. 3, with component number on the X-axis and the channel on the Y-axis), and its own time series that runs through all channels, across the entire sample of the EEG recording.

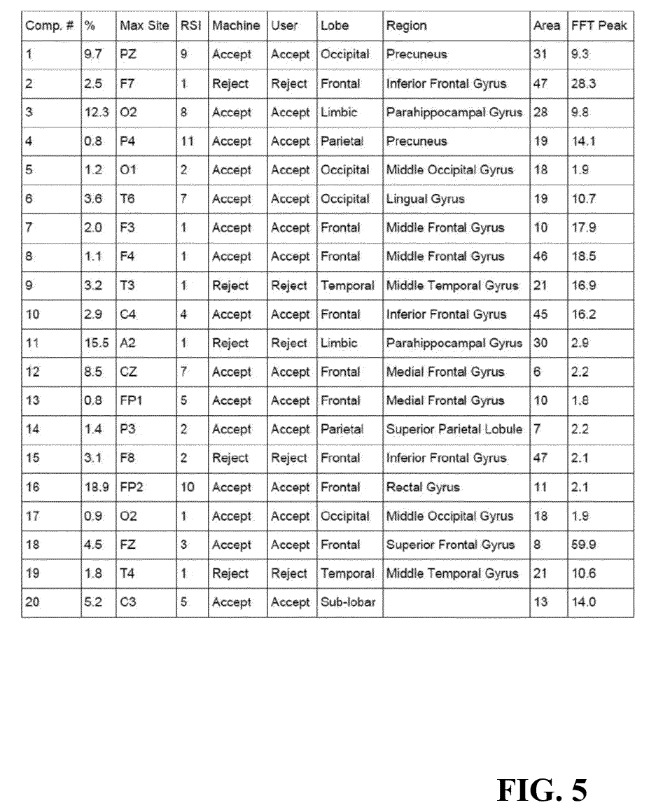

FIG. 5 shows a list of components and characteristics of the components C1-C20 that are observable after the ICA process including the percentage of total EEG energy that is contained in the component (%), the surface site at which it is maximum (Max Site), relative sharpness index (RSI), the machine (Machine) and user (User) decisions whether to accept the component for further processing, the standardized Low Resolution Electromagnetic Tomography (sLORETA) localized lobe (Lobe), brain region (Region), Brodmann area (Area), and Fast Fourier transform graph (FFT) peak energy (FFT Peak). These characteristics can assist the user in classifying components in categories such as artifact, posterior dominant rhythm (PDR), midline theta, frontal sources, and other classifications.

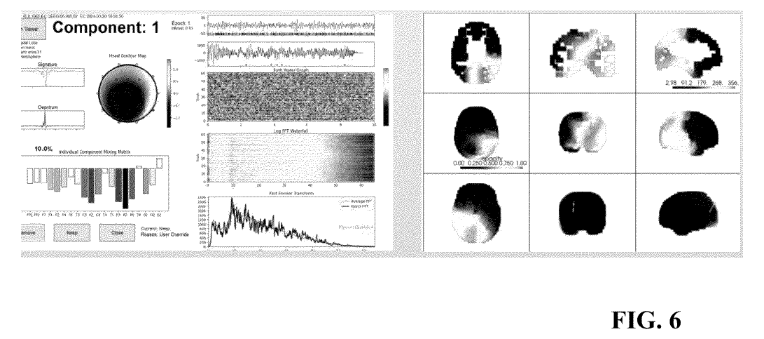

FIGS. 6-9 show analyses related to the application of the ICA decomposition both before and after sLORETA analysis for selected components, along with their associated deconvolutions and recovered point processes.

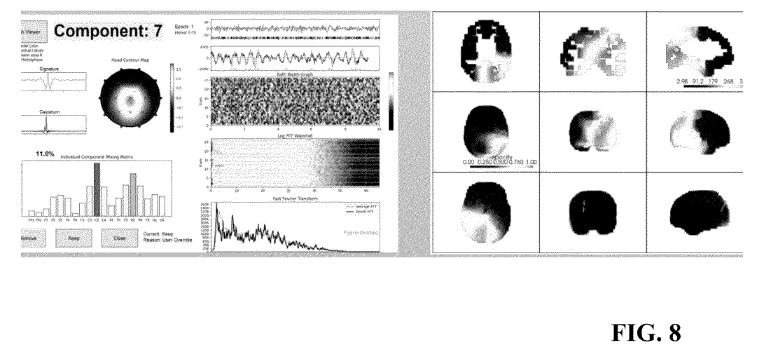

In one embodiment, the ICA can identify unique posterior dominant rhythm sources. For example, components 1 and 7, corresponding to FIGS. 6 and 8, show posterior dominant rhythms coming from different sources and with slightly different spectral distributions. These are individual characteristics for that patient.

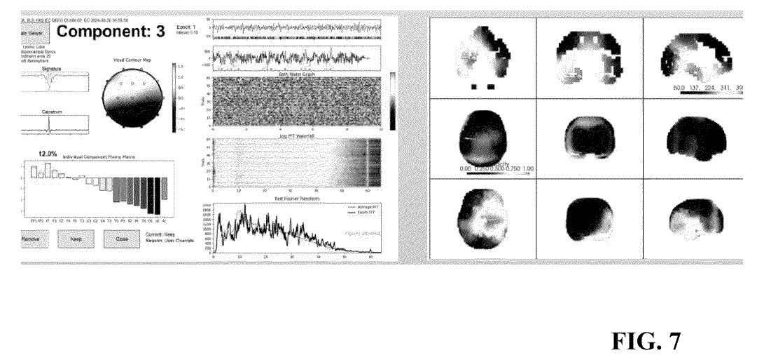

In one embodiment, the processing system 300 using ICA can identify mixed rhythms from Brodmann Area 6. For example, in component 3, corresponding to FIG. 7, shows a mixed rhythm arising from the parahippocampal gyrus. The mixed rhythm contains wide range of frequencies encompassing essentially the entire range of EEG frequencies.

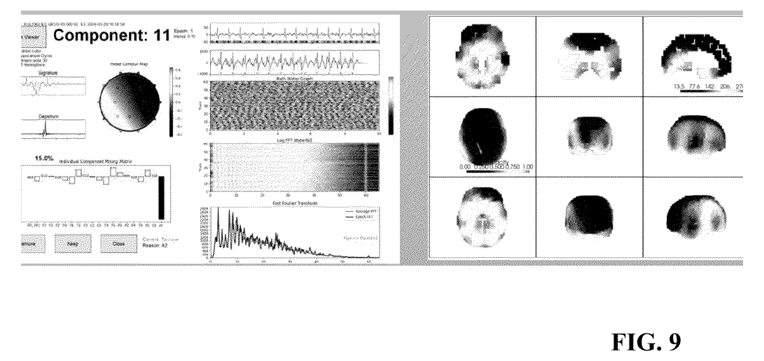

In one embodiment, the processing system 300 using ICA can extract and deconvolve EEG or electrocardiogram (EKG) waves. For example, component 11, corresponding to FIG. 9, shows an EKG artifact that is found to be maximum at A2. The disclosed methods and ICA correctly identifies the signature with the distinct appearance of a P-QRS-T wave morphology, confirming that the disclosed methods are accurate to signal morphology. The disclosed methods also accurately pinpoints the heart beats using the template matching method. In some embodiments, heart rate variability statistics can be computed from this data. Additionally, the source localization identifies the source at the base of the neck, which is where the field actually appears to arise from as volume conduction. The timing statistics for these components are revealed in the points depicted on the example 10-second time-series plot. This plot is created by using a template-matching method that finds occurrences of the signature in the raw component signal across time.

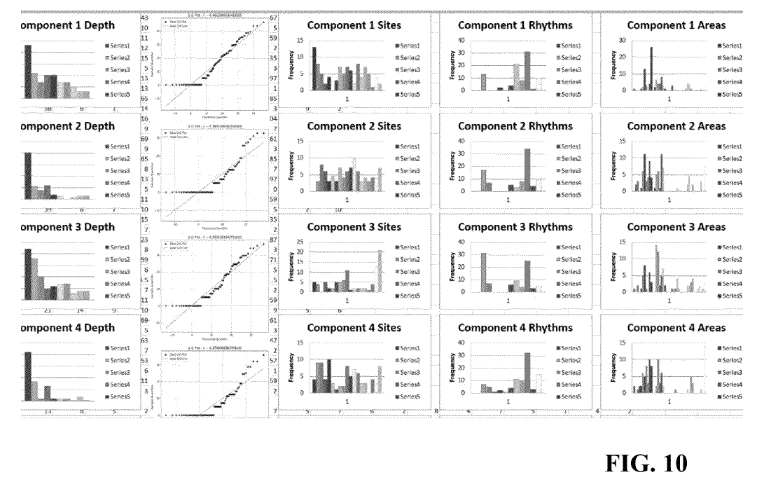

FIG. 10 shows additional data that can be determined through the processing system 300 using ICA by classifying each component by type (e.g., PDR, midline theta, etc.) and recording their occurrence in each EEG. For example, some EEGs may have one PDR generator, while others may have two or more independent generators. These may produce individual differences in the EEG. In an embodiment, these components can provide the basis for a new database that can include such data as the depth of each component, its surface locations, frequency content, sLORETA location, classification, time statistics, and other information, so that each component source can be classified and rated in comparison to data obtained from numerous EEG recordings.

It is noted that the disclosed systems and methods do not use z-scores to create the localization or images. In one embodiment, all data is processed as raw EEG and no database is used in these computations. It is possible to create z-scores from this data, leading to a new kind of database. Rather than analyzing the EEG using strictly Fourier methods, which reduces the signal to a set of frequencies, the disclosed systems and methods uses ICA as a reverse source localization to removes volume conduction. The disclosed systems and methods break down EEGs as a series of events where each component has a mixture of frequencies, and its own fingerprint or “signature.” These signatures may occur in the EEG on an intermittent basis, providing more of a series of chirps than a frequency.

In one embodiment, the viewing an analysis tools of the disclosed systems and methods may include an EEG viewer displaying, for example, the 10-second epochs, an individual FFT and averaged FFT graph, a “bathwater” graph of the component amplitudes color-coded across time for all epochs, and inverse of the averaged FFT, and/or the “periodogram” of the periods that the component is present in the original signal. When constructing a database, the statistics of this gating function can become one set of metrics that can be characterized in a typical database. The database may be constructed to include classification for each component for each individual, peak frequencies, sLORETA depth, and/or statistics describing the time behavior of each component, for example. This creates a new type of metric analysis that reflects underlying stable generators and time behavior in an individualized basis and can provide neurofeedback based upon the template matching. Knowing the shape of each event, the disclosed systems and methods can provide real-time cross-correlation and reward occurrences of each transient.

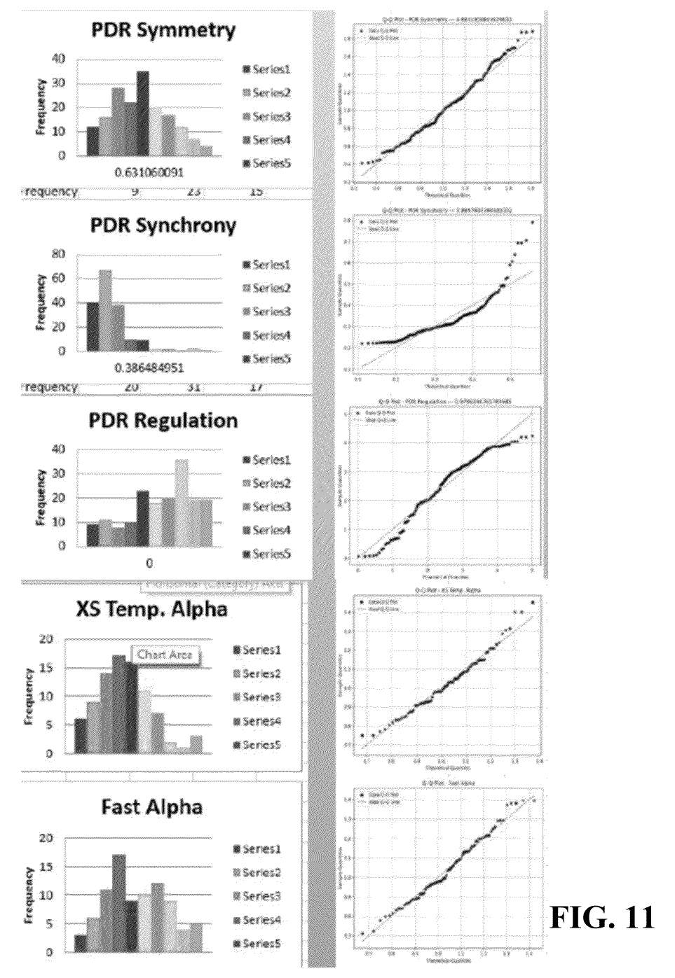

FIG. 11 shows characteristics from a database of EEGs that were analyzed by a board certified clinical neurophysiologist and QEEG Diplomates to comment on the quality of EEGs submitted and to make any relevant clinical observations regarding severe abnormalities or EEG quality problems. The observations were provided as a database from two sets of data, which have samples sizes of 95 and 190, respectively. The database represents what is seen in a clinical EEG service and were analyzed with regard to what a visual inspection of the EEG might indicate. In an example, the larger database was found to produce slightly wider acceptance bands, but did not affect the resulting z-scores.

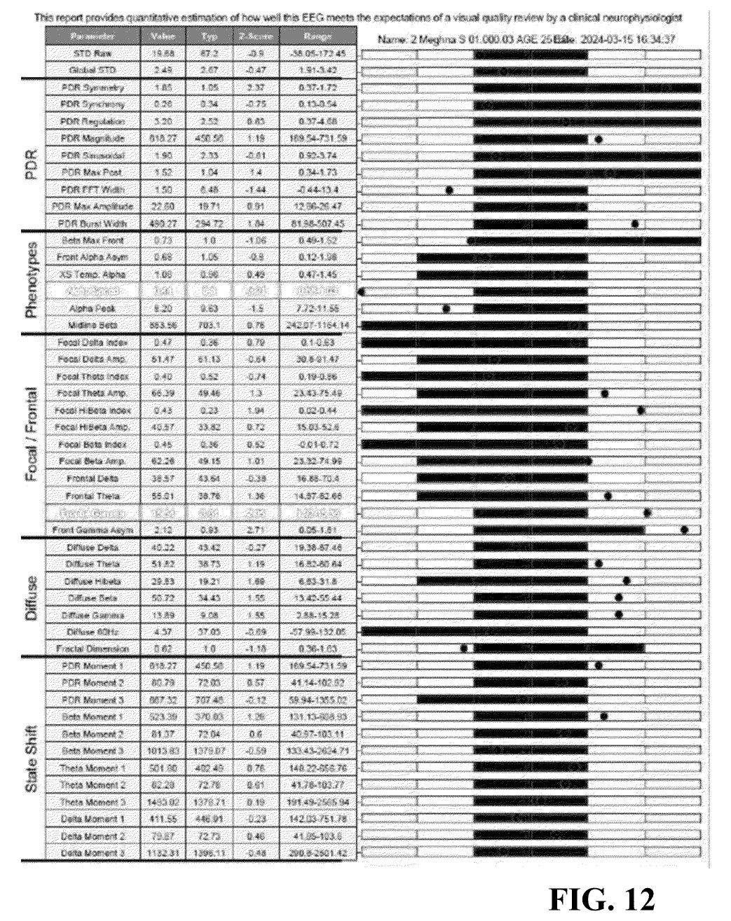

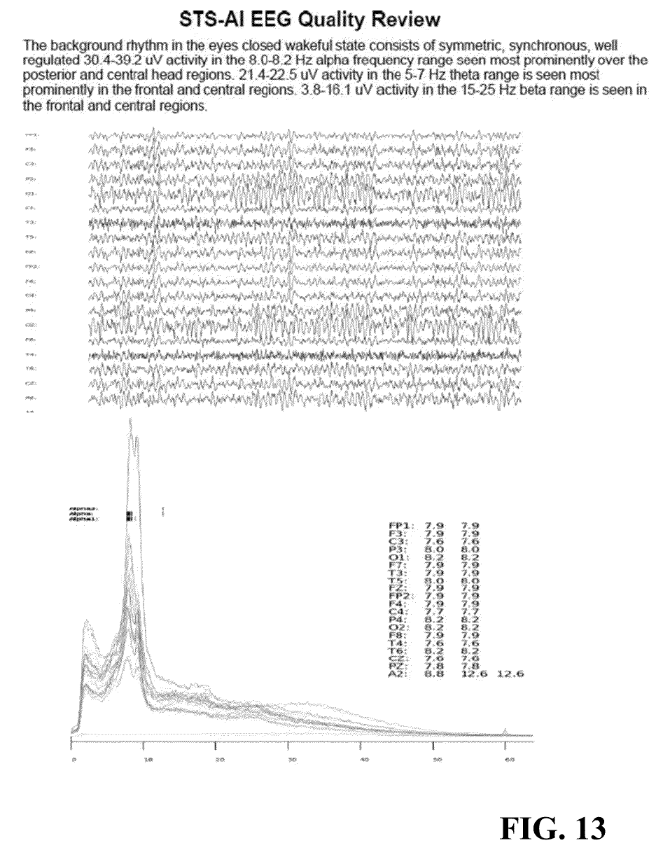

FIGS. 12-13 show a qualitative estimation of how the disclosed systems and methods analyze EEGs and how the analysis and results compares to the expectations of a visual quality review by a clinical neurophysiologist. The disclosed systems and methods may pre-screen EEGs that have not yet been inspected or artifacted, to determine how well they fit into a “typical” type of recording. The disclosed systems and methods may further identify phenotypes that may be evident. The a qualitative estimation can provide insight into clinical referrals based on EEGs and prepare the recording for QEEG analysis (e.g., as a “pre-Q” or even a “pre-pre-Q” analysis). As shown in FIG. 12, the disclosed systems and methods can provide a report composed and formatted to replicate a physician’s quality review and can comparatively determine accuracy based on the quantitative findings derived from the metric analysis.

The disclosed systems and methods may comprise computer readable instructions as herein described which use digital signal processing to simulate a physician’s a visual inspection of an EEG. The disclosed systems and methods may define a set of reasonably informed metrics or input parameters based on one or more of the posterior dominant rhythms, amplitude foci and magnitudes, time course of various metrics, and the like. In an embodiment, the ICA may be applied to remove eye artifact. The disclosed systems and methods may analyze a series of 10-second epochs, providing a 0.1 Hz resolution for the FFT having a frequency content, up to 64 Hz. The disclosed systems and methods may further analyze a multitude of EEGs to compute the population statistics of the input samples (similar to QEEG analysis).

In some embodiments, the disclosed systems and methods may provide automated screening of EEGs without the use of artificial intelligence. It is noted that the disclosed systems and methods can use z-score to quantify EEG properties, but are not the same as QEEG. As a form of quality control, and as an assist to a clinical neurophysiologist, disclosed systems and methods may provide automation in reading EEGs and an aid to visual inspection, but may not replace any of the human tasks required for sound EEG analysis, or preparation for further processing. In addition to metrics reflecting relative amplitudes and distributions of key metrics, the disclosed systems and methods analyze “moments” which reflect the total size of a component amplitude, as well as its distribution within and across the recording session. Extreme moments tend to reflect drowsiness and other changes across a recording, showing changes across time. These may be similar but not identical to reliability measures such as splitting half and test-retest reliability.

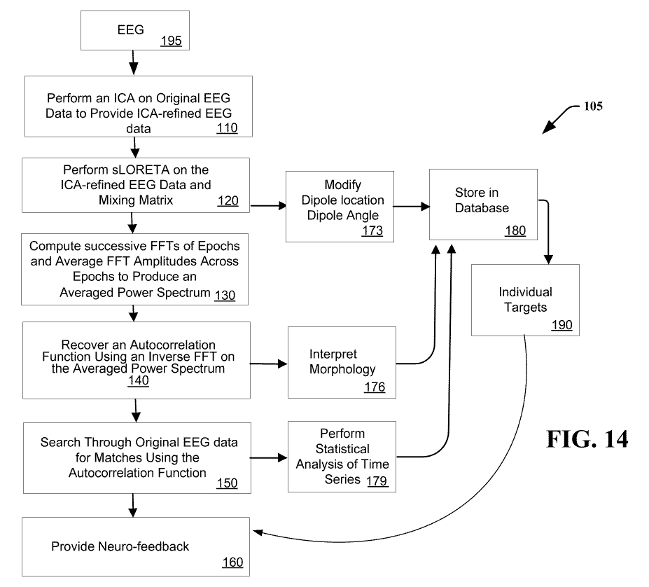

Turning to FIG. 14, disclosed is a method for analyzing and evaluating electroencephalograms (EEGs) 105, for example, as an EDF file or live EEG. Similar to method 100, method 105 may include the steps as shown in FIG. 14. In one embodiment, a live EEG 195 is input into the system, and the system performs independent component analysis (ICA) on original EEG data to provide ICA-refined EEG data (step 110). For example, in one embodiment, the ICA step 110 may be used to remove eye activity. The method 105 further perform standardized Low Resolution Electromagnetic Tomography (sLORETA) on the ICA-refined EEG data and mixing matrix (step 120). In one embodiment, the sLORETA projection of the mixing matrix may be applied to each component of the ICA-refined data to estimate the source location and orientation. After performing sLORETA, the method 105 may use the results and analysis of step 120, to modify/adjust the dipole location and dipole angle (step 173). In some embodiments, this modifying step is skipped, or no modification is performed. The dipole location and angle can be stored in database (step 180).

Further, the method 105 may compute successive Fast Fourier transforms (FFTs) of epochs of the EEGs to and average FFT amplitudes across epochs to produce an averaged power spectrum (steps 130). In an embodiment, the epochs may be 10 second epochs across the entire EDF file. Next, the method may recover an autocorrelation function using an inverse FFT on the averaged power spectrum (step 140). In one embodiment, morphology is interpreted (step 176) from the results and analysis of step 140. The method 105 may search through original EEG data for matches using an autocorrelation function (step 150). In one embodiment, the method may perform statistical analysis of time series (step 179) on the results and analysis of step 150. In some embodiment, both the information from the interpreted morphology (step 176) and statistical analysis of time series (step 179) can be stored in database (step 180).

In one embodiment, individual targets may be determined and assessed from the database and analysis of one or more of the prior steps, which can provide real-time neuro-feedback (step 160). For example, the matches between one or more (or all of) the autocorrelation function from step 150, the individual targets determined at step 190, the original EEG data, and live EEG data 195 may be marked as events and used for real-time neuro feedback at step 160. The type of the event, the shape of the event, or other characteristics of the event can be used to determine the type of neuro-feedback required. This neuro-feedback can be present to the user using a display or other output devices, such as lights and buzzers.

As with method 100, in one embodiment, method 105 may be used deconvolve EEG data into individual sources and patterns using various analysis tools described herein. The detection and isolation of singular events and the association of the events to the particular times that they occur can allow insight into the morphology of the waves, and the exact timing of the brain events, including effects that change across time.

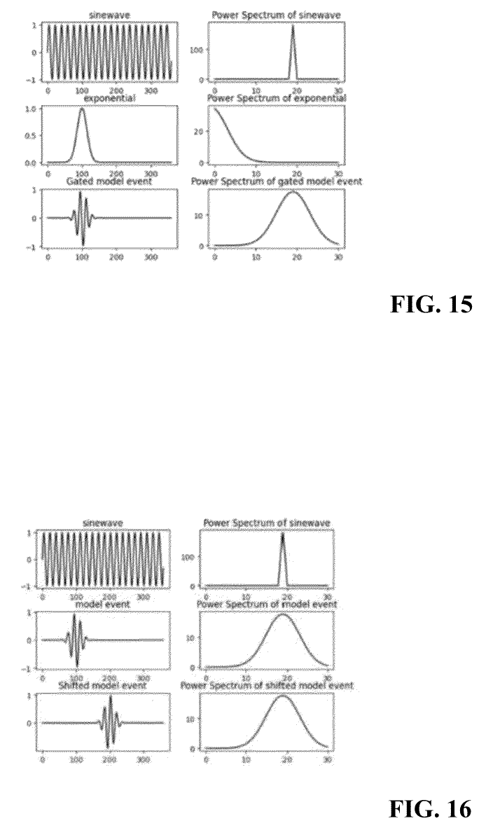

FIGS. 15-21 illustrate example methods of determining different model events. These determinations can be used by the above systems and methods to deconvolute EEG signals and extract individual components. The example methods are illustrated using 10 second epochs but this timing can be more or less depending on the source EEG, the component, the characteristics of the component, or the like.

FIGS. 15-16 shows analysis from creating a wavelet by gating a sinewave, which convolves the sinewave power spectrum with the gate power spectrum and that the power spectrum of wavelet is independent of phase. Using this technique, the system and method can detect embedded wavelets in the 10 second epochs, as described above. In an embodiment, foundations of an event model for EEG power spectra may be provided or developed and analyzed. For instance, regarding FIG. 15, the sinewave convolves the sinewave power spectrum with the gate power spectrum. Particularly, the sinewave has a single peak in its power spectrum and the model event also has its characteristically shaped power spectrum. When the sinewave is gated, the systems can determine a spread spectrum centered on the frequency of the sinewave. This is the characteristic power spectrum of the event.

Additionally, regarding FIG. 16, the power spectrum of wavelet is independent of phase. Particularly, the power spectrum of the event does not depend on its location, i.e., is phase insensitive. For instance, the sinewave has a single peak in its power spectrum. When the sinewave is gated, the systems can determine a spread spectrum centered on the frequency of the sinewave. Here, the power spectrum of the event does not depend on its location, i.e., phase insensitive. In some embodiments, a Fourier transform is used to convert from time to frequency assuming everything is a sinewave. In one embodiment, non-sinusoidal signals may have a fundamental and harmonics based on the wave shape. In other embodiments, the power spectrum may be linear and/or adding inputs may add to their power spectra.

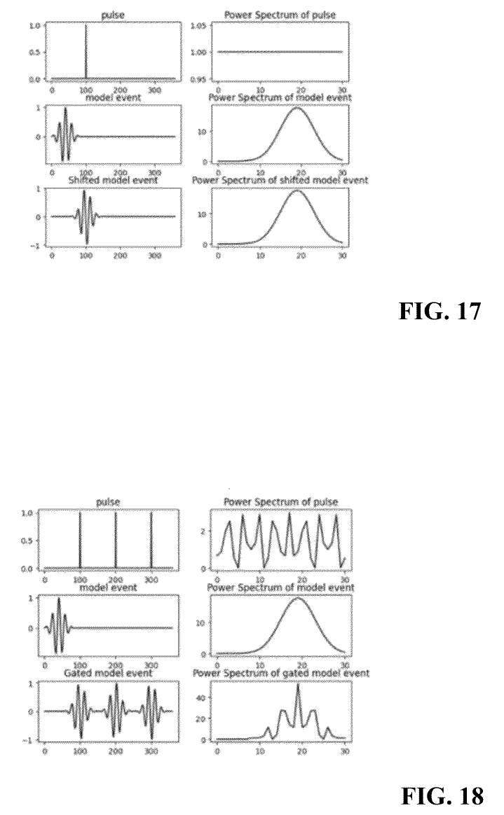

FIGS. 17-20 shows analysis of pulses and pulse trains. Regarding FIG. 17, a pulse convolved with an event may place that event in time and the power spectrum may not change. Particularly, the single pulse has a broadband power spectrum, i.e., contains all frequencies. The event has its characteristically shaped power spectrum independent of timing, i.e., phase. The system and method can determine the power spectrum of the even regardless of its phase by multiplying the spectra.

Regarding FIG. 18, a series of wavelets (convolving pulses with wavelet) may result in the product of the power spectra. Particularly, the pulse series has a rippled power spectrum pattern. The event has its characteristically shaped power spectrum. The systems and method can convolve the pulses with the model event to produce the train of events in the EEG. The systems and method can determine a rippled power spectrum with amplitudes based on the event when the pulses are multiplied.

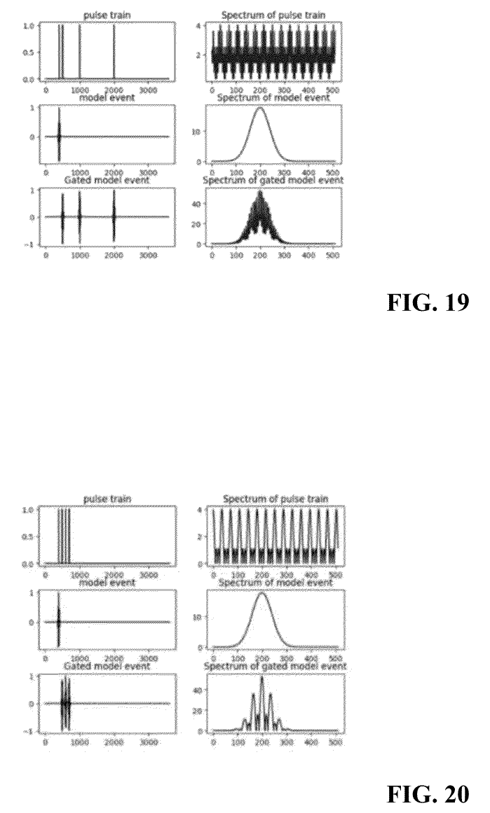

Regarding FIG. 19, the power spectrum may be a series of irregularly timed events. Particularly, the pulse train has its own spectrum consisting of “spikes” in the frequency domain. The spikes can be multiplied by the power spectrum of a single pulse to create the spectrum of the event train. If the wavelets are irregularly spaced, they will produce a “fuzzy” power spectrum. The overall envelop of the power spectrum reflects the shape of one event, with the ripples representing the timing of the pulse train.

Regarding FIG. 20, the power spectrum may have ripples that represent the timing of the events. Particularly, the pulse train has its own spectrum consisting of “spikes” in the frequency domain. The spikes can be multiplied by the power spectrum of a single pulse to create the spectrum of the event train. If the wavelets are regularly space, the multiplication will produce a “ripples” power spectrum. The overall envelope of the power spectrum reflects the shape of one event and the ripples represent the timing of the pulse train.

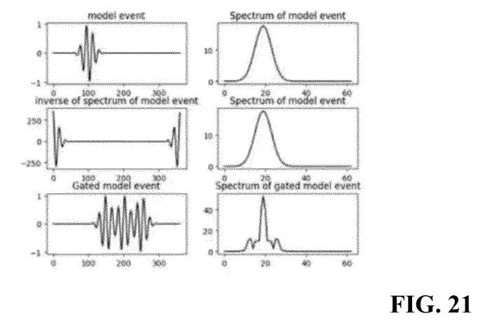

Regarding FIG. 21, the inverse of the spectrum may be the autocorrelation of the model event. Particularly, the model event has its characteristics power spectrum. The systems and methods can determine the autocorrelation of the original signal if a Fourier Transform is applied to the power spectrum. If multiple events are combined in a train, the power spectrum becomes a notched version of the event spectrum.

These methods above can be used to deconvolute the EEG signals and determine individual components of the EEG signal. For instance, these methods can be applied to the 10 second epochs to detect the embedded wavelets. In some embodiments, a random series of wavelets may have a spread power spectrum and/or averaging power spectra may produce an estimate of the wavelets.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments.

For example, changes may be made in the function and arrangement of elements discussed without departing from the scope of the disclosure. Various examples may omit, substitute, or add various procedures or components as appropriate. For instance, the methods described may be performed in an order different from those described, and various steps may be added, omitted, or combined. Also, features described with respect to some examples may be combined in some other examples. For example, an apparatus may be implemented, or a method may be practiced using any number of the aspects set forth herein. In addition, the scope of the disclosure is intended to cover such an apparatus or method practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to "at least one of' a list of items refers to any combination of those items, including single members. As an example, "at least one of: a, b, or c" is intended to cover a, b, c, a-b, a-c, b-c, and a-b-c, as well as any combination with multiples of the same element (e g .. , a-a ' a-a-a ' a-a-b ' a-a-c ' a-b-b ' a-c-c ' b-b ' b-b-b ' b-b-c ' c-c ' and c-c-c or any other ordering of a, b, and c ).

As used herein, the term "determining" encompasses a wide variety of actions. For example, "determining" may include calculating, computing, processing, deriving, investigating, looking up (e.g., looking up in a table, a database, or another data structure), ascertaining, and the like. Also, "determining" may include receiving ( e.g., receiving information), accessing ( e.g., accessing data in a memory), and the like. Also, "determining" may include resolving, selecting, choosing, establishing, and the like.

The methods disclosed herein comprise one or more steps or actions for achieving the methods. The method steps or actions may be interchanged with one another without departing from the scope of the claims. In other words, unless a specific order of steps or actions is specified, the order or use of specific steps or actions may be modified without departing from the scope of the claims. Further, the various operations of methods described above may be performed by any suitable means capable of performing the corresponding functions. The means may include various hardware or software component(s) or module(s), including, but not limited to a circuit, an application specific integrated circuit (ASIC), or processor. Generally, where operations are illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean "one and only one" unless specifically so stated, but rather "one or more." Unless specifically stated otherwise, the term "some" refers to one or more.

What is claimed is:

11 . A method of analyzing electroencephalogram signals, comprising:
capturing the electroencephalogram (EEG) signals from a plurality of sensors;
performing independent component analysis (ICA) on the electroencephalogram signals to provide ICA-refined electroencephalogram data;
performing standardized Low Resolution Electromagnetic Tomography (sLORETA) on the ICA-refined electroencephalogram data;
computing successive Fast Fourier Transforms (FFT) of epochs and averaging FFT amplitudes of the epochs to provide an averaged power spectrum;
computing an inverse FFT on the averaged power spectrum and using autocorrelation to find matches with the electroencephalogram signals, and
determining a plurality of events based on matches of the autocorrelation and the electroencephalogram signals.

2. The method of claim 1, further comprising:
displaying the plurality of determined events.

3. The method of claim 1, wherein performing ICA produces a plurality of individual components.

4. The method of claim 3, wherein each of the plurality of individual components has a complete frequency spectrum and an amplitude for every channel of the electroencephalogram signals.

5. The method of claim 3, further comprising:
displaying the plurality of components,
wherein the display includes a percentage of total EEG energy contained in the component, a surface site at which it is maximum, a sLORETA localized lobe, a brain region, a Brodmann area, and a FFT peak energy for each component of the plurality of components.

6. The method of claim 1, further comprising:
providing neuro-feedback based on the determined plurality of events.

7. The method of claim 6, wherein the neuro-feedback is based on the shape of the determined plurality of events.

8. A system for analyzing electroencephalogram signals, comprising:
one or more computer memory units for storing computer instructions; and one or more processors operatively coupled to the one or more computer memory units, the one or more processors configured to perform the operations of:
capturing the electroencephalogram signals from a plurality of sensors;
performing independent component analysis (ICA) on the electroencephalogram signals to provide ICA-refined electroencephalogram data;
performing standardized Low Resolution Electromagnetic Tomography (sLORETA) on the ICA-refined electroencephalogram data;
computing successive Fast Fourier Transforms (FFT) of epochs and averaging FFT amplitudes of the epochs to provide an averaged power spectrum;
computing an inverse FFT on the averaged power spectrum and using autocorrelation to find matches with the electroencephalogram signals, and
determining a plurality of events based on matches of the autocorrelation and the electroencephalogram signals.

9. The system of claim 8, further comprising:
a display configured to display the plurality of determined events.

10. The system of claim 8, wherein performing ICA produces a plurality of individual components.

11. The system of claim 10, wherein each of the plurality of individual components has a complete frequency spectrum and an amplitude for every channel of the electroencephalogram signals.

12. The system of claim 10, wherein the display is configured to display the plurality of individual components in a graph or table, and
wherein the graph or table includes a percentage of total EEG energy contained in the component, a surface site at which it is maximum, a sLORETA localized lobe, a brain region, a Brodmann area, and a FFT peak energy for each component of the plurality of components.

13. The system of claim 10, further comprising:
an output configured to provide neuro-feedback based on the determined plurality of events.

14. The system of claim 13, wherein the neuro-feedback is based on the shape of the determined plurality of events.
